(19) United States
(12) Patent Application Publication
(10) Pub. No.: US 2023/0341302 A1
(43) Pub. Date: Oct. 26, 2023

(54) MICRO PRE-CONCENTRATOR HAVING CERAMIC-POLYMER COMPOSITE WITH 3D NANO-SHELL STRUCTURE AND METHOD FOR MANUFACTURING THE SAME

(71) Applicant: Korea Advanced Institute of Science and Technology, Daejeon (KR)

(72) Inventors: Seokwoo Jeon, Daejeon (KR); Byungyoon Chung, Daejeon (KR); Kisun Kim, Daejeon (KR)

(73) Assignee: Korea Advanced Institute of Science and Technology, Daejeon (KR)

(21) Appl. No.: 18/302,248

(22) Filed: Apr. 18, 2023

(30) Foreign Application Priority Data
Apr. 26, 2022 (KR) 10-2022-0051312

(51) Int. Cl. G01N 1/40

(52) U.S. Cl. CPC G01N 1/405

(57) ABSTRACT

A disclosed micro pre-concentrator includes a base substrate having a trench, and a 3D nano-shell composite disposed in the trench and having pores that are ordered and connected to each other. The 3D nano-shell composite includes a 3D nano-shell support defined by a thin film extending three-dimensionally and a polymeric layer formed along a surface of the 3D nano-shell support. The micro pre-concentrator may increase adsorption amount of gas. In addition, since it may be uniformly heated in a short time, it is possible to release gas at a high density in a short time.

### CROSS-REFERENCE TO RELATED APPLICATION

This application claims priority to and benefits of Korean Patent Application No. 10-2022-0051312 under 35 U.S.C. § 119 filed on Apr. 26, 2022 in the Korean Intellectual Property Office, the entire contents of which are incorporated herein by reference.

### BACKGROUND

### 1. Technical Field

The present invention relates to a pre-concentrator. More particularly, the present invention relates to a pre-concentrator that has a three-dimensional (3D) nano-shell structure and can be used for detecting volatile organic compounds, and a method for manufacturing the pre-concentrator.

### 2. Description of the Related Art

Volatile organic compounds (VOCs) may badly affect human bodies, for example, by forming particulate matters in the air, generating odor or reacting with light to generate ozone. Conventional detectors may hardly measure or detect gaseous components with low concentration in the air due to low detection ability thereof. In order to the above-mentioned problem, a pre-concentrator may be used for concentrating gaseous components before the gaseous components are provided to detectors.

Recently, as need for a portable detector is increased, requirement for developing a micro pre-concentrator that may be applicable for a portable detection system is being increased. For example, a micro pre-concentrator may be fabricated by using a micro-column based on a MEMS process and absorbent.

In a conventional micro pre-concentrator, a porous material is filled in a micro pre-concentrator thereby forming a porous structure having a large specific surface area and capable of maximizing absorption/desorption efficiencies between gaseous components and the porous structure. However, because a convention porous structure is a random porous structure with a micron scale, it may be difficult that gaseous molecules smoothly flow when absorbed and desorbed. Furthermore, because heat is hardly provided uniformly to the porous structure, desorption failure may be partially caused thereby reducing concentration effect.

### Patent Literature

(1) PCT International Patent Application No. PCT/EP2015/063293

(2) U.S. Pat. No. 9,316,623

(3) Korean Granted Patent 10-2112031

### Non-patent Literature

(1) Anal. Chem. 2012, 84, 6336

(2) Lab Chip, 2012, 12, 717

(3) Lab Chip, 2013, 13, 818

### SUMMARY

One object of the present invention is to provide a micro pre-concentrator including a ceramic-polymer composite having a 3D nano-shell structure.

Another object of the present invention is to provide a method of manufacturing the micro pre-concentrator.

According to an embodiment of the present invention, a micro pre-concentrator includes a base substrate having a trench, and a 3D nano-shell composite disposed in the trench and having pores that are ordered and connected to each other. The 3D nano-shell composite includes a 3D nano-shell support defined by a thin film extending three-dimensionally and a polymeric layer formed along a surface of the 3D nano-shell support.

In an embodiment, a thickness of the 3D nano-shell composite is equal to or more than 20 μm.

In an embodiment, a shell thickness of the 3D nano-shell support is 10 nm to 50 nm, and a thickness of the polymeric layer is 50 nm to 100 nm.

In an embodiment, the 3D nano-shell support includes a ceramic.

In an embodiment, the polymeric layer includes a porous polymer.

In an embodiment, the micro pre-concentrator further includes a heating member disposed under the base substrate.

According to an embodiment of the present invention, a method of manufacturing a micro pre-concentrator includes forming a 3D porous template in a trench of a base substrate, forming a 3D nano-shell support along a surface of the 3D porous template, removing the 3D porous template, coupling a cover substrate to the base substrate such that the cover substrate covers the trench after removing the 3D porous template, and providing a polymer solution in the trench, after coupling the cover substrate to the base substrate, to form a polymeric layer having a 3D structure along a surface of the 3D nano-shell support.

In an embodiment, forming of the 3D porous template includes forming an adhesive film, of which at least a portion is disposed in the trench, forming a photosensitive film in the trench, the photosensitive film contacting the adhesive film, irradiating a three-dimensionally distributed light onto the photosensitive film through a phase mask, and developing the photosensitive film.

In an embodiment, the adhesive film covers an entire portion of a bottom surface of the trench, and the photosensitive film is formed on the adhesive film.

In an embodiment, the adhesive film covers a side surface of the trench, and a lower surface of the photosensitive film contacts a bottom surface of the trench, and a side surface of the photosensitive film contacts the adhesive film.

In an embodiment, the 3D nano-shell support is formed through deposition and includes a ceramic.

In an embodiment, the polymer solution includes 0.5 wt % to 2 wt % of a porous polymer.

In an embodiment, a thickness of the adhesive film is 0.5 μm to 2 μm.

According to embodiments of the present invention, a 3D nano-shell composite is used for gas concentration. Since the 3D nano-shell composite has a network structure, it has a large surface area, and may transfer heat uniformly and fast, and has low weight due to high porosity. Therefore, it is possible to increase adsorption amount of gas. In addition, since the 3D nano-shell composite may be uniformly heated in a short time, it is possible to release gas at a high density in a short time. Thus, it is possible to increase the concentration performance of the micro pre-concentrator.

### BRIEF DESCRIPTION OF THE DRAWINGS

FIG. 1 is a plan view illustrating a pre-concentrator according to an embodiment of the present invention.

FIG. 2 is a cross-sectional view taken along the line of I-I′ in FIG. 1.

FIGS. 3, 4, 5, 6, 7 and 8 are cross-sectional views illustrating a method of manufacturing a pre-concentrator according to an embodiment of the present invention.

FIG. 9A is a perspective view schematically illustrating a step of forming a 3D nano-shell composite from a 3D porous template in a method of manufacturing a pre-concentrator according to an embodiment of the present invention.

FIG. 9B is a cross-sectional view schematically illustrating a step of forming a 3D nano-shell composite from a 3D porous template in a method of manufacturing a pre-concentrator according to an embodiment of the present invention.

FIG. 10 is a plan view illustrating a step of injecting a polymer solution into a trench in a method of manufacturing a pre-concentrator according to an embodiment of the present invention.

FIGS. 11 and 12 are cross-sectional views illustrating a trench in which an adhesive film and a photosensitive film are formed in a method of manufacturing a micro pre-concentrator according to embodiments of the present invention.

FIG. 13 is a scanning electron microscope (SEM) picture of a 3D nano-shell support obtained after removing a polymeric template and an adhesive film in Example 1.

FIG. 14 shows SEM pictures of 3D nano-shell composites obtained according to concentrations of polymer (Tenax).

FIG. 15 is an energy dispersive X-ray spectroscopy (EDS) image of the 3D nano-shell composite (Tenax concentration: 1 wt %) of Example 1.

FIG. 16 shows schematic diagrams of experimental models for evaluating the concentration performance of a micro pre-concentrator according to an embodiment of the present invention.

FIG. 17 shows graphs showing chromatographic results and peak areas for mixed samples measured through the basic loop.

FIG. 18 shows graphs showing chromatographic results and peak areas for mixed samples measured through the empty trench.

FIG. 19 shows graphs showing chromatographic results and peak areas for mixed samples measured through the 3D nano-shell support (thickness: 25 μm) not coated with the polymer in Example 1.

FIG. 20 shows graphs showing chromatographic results and peak areas for mixed samples measured through the 3D nano-shell composite (thickness: 25 μm, polymer concentration: 1 wt %) coated with the polymer in Example 1.

FIG. 21 is a graph showing the concentration ratios of the 3D nano-shell composite coated with the polymer in Example 1 according to the thickness of the 3D nano-shell composite.

### DETAILED DESCRIPTION OF THE EMBODIMENTS

Various embodiments will be described more fully hereinafter with reference to the accompanying drawings, in which some embodiments are shown. The present invention may, however, be embodied in many different forms and should not be construed as limited to the embodiments set forth herein. Rather, these embodiments are provided so that this description will be thorough and complete, and will fully convey the scope of the present invention to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third etc. may be used herein to describe various elements, components, regions, layers and/or parts, these elements, components, regions, layers and/or parts should not be limited by these terms. These terms are only used to distinguish one element, component, region, layer or part from another region, layer or part. Thus, a first element, component, region, layer or part discussed below could be termed a second element, component, region, layer or part without departing from the teachings of the present invention.

The terminology used herein is for the purpose of describing particular embodiments only and is not intended to be limiting of the present invention. As used herein, the singular forms “a,” “an” and “the” are intended to include a plurality of forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

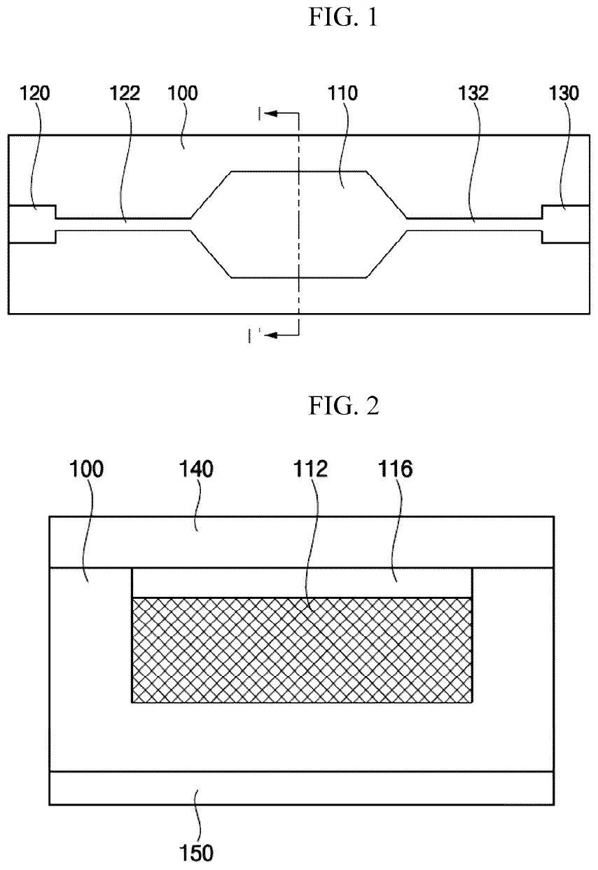

FIG. 1 is a plan view illustrating a pre-concentrator according to an embodiment of the present invention. FIG. 2 is a cross-partial view taken along the line of I-I′ in FIG. 1.

Referring to FIGS. 1 and 2, a pre-concentrator according to an embodiment may include a base substrate 100. A trench may be formed at the base substrate 100. The trench may include a concentration part 110, an injection part 120 and a discharge part 130. Additionally, the trench may include an injection channel 122 for connecting the concentration part 110 to the injection part 120 and a discharge channel 132 for connecting the concentration part 110 to the discharge part 130.

For example, the base substrate 100 may include silicon, glass, quartz or the like.

A 3D nano-shell composite 112 may be disposed in the concentration part 110. The 3D nano-shell composite 112 may have ordered pores three-dimensionally connected to each other. The 3D nano-shell composite 112 may include a 3D nano-shell support including ceramic and a polymeric layer coated on the 3D nano-shell support and including a polymer. The polymeric layer extends along a surface of the 3D nano-shell support thereby forming a 3D structure.

For example, the 3D nano-shell support may include a metal oxide or a metal nitride of a metal such as Zn, Al, Ni, Mo, Co, Sn, Fe, W, Ti, Mn, Zr, Cu or the like. For example, the 3D nano-shell support may include aluminum oxide (Al₂O₃), titanium oxide (TiO₂), zirconium oxide (ZrO₂), zinc oxide (ZnO), titanium nitride (TiN), or combinations thereof.

The polymer may be a porous polymer having a bulky structure. For example, the porous polymer may include poly(2,6-diphenyl-p-phenylene oxide) or the like. Particularly, commercially available porous polymers such as Tenax® TA, Tenax® GR, Carbosieve®, Carbopack®, HayeSep® or the like may be used for the porous polymer. The porous polymer may effectively absorb VOCs. However, embodiments of the present invention are not limited thereto, and the polymeric layer may include various materials depending on a target material.

In an embodiment, the 3D nano-shell composite 112 may have a film shape, and a thickness of the 3D nano-shell composite 112 along a vertical direction may be about 5 μm to about 500 μm. Preferably, a thickness of the 3D nano-shell composite 112 may be equal to or more than about 20 μm. For example, a thickness of the 3D nano-shell composite 112 may be about 20 μm to about 100 μm, or may be about 20 μm to about 50 μm. When a thickness of the 3D nano-shell composite 112 is too small, concentration efficiency may be reduced. When a thickness of the 3D nano-shell composite 112 is excessively large, manufacturing reliability or mass transfer efficiency of the 3D nano-shell composite 112 may be reduced.

In an embodiment, a shell thickness of the 3D nano-shell support including ceramic may be about 10 nm to about 100 nm. When a shell thickness of the 3D nano-shell support is too small, mechanical properties of the 3D nano-shell composite 112 may be reduced. When a shell thickness of the 3D nano-shell support is excessively large, specific surface area and concentration efficiency of the 3D nano-shell composite 112 may be reduced. For example, a shell thickness of the 3D nano-shell support including ceramic may be about 10 nm to about 50 nm.

For example, a thickness of the polymeric layer may be about 20 nm to about 200 nm. In an embodiment, a thickness of the polymeric layer may be about 50 nm to about 100 nm. When a thickness of the polymeric layer is too small, uniformity thereof may be reduced. When a thickness of the polymeric layer is excessively large, specific surface area and concentration efficiency of the 3D nano-shell composite 112 may be reduced.

In an embodiment, the micro pre-concentrator may include a cover member 140 coupled to the base substrate 100. The cover member 140 and the 3D nano-shell composite 112 may be separated from each other such that a gap 116 may be formed therebetween. The gap 116 may be used as a path for gaseous components which may be provided into the pre-concentrator.

When a gaseous analyte enters the concentration part of the micro pre-concentrator, the 3D nano-shell composite 112 may selectively absorb components of the analyte. The 3D nano-shell composite 112 may be heated to desorb the absorbed components. For example, the micro pre-concentrator may further include a heating member 150 combined with the base substrate 100. For example, the heating member 150 may be disposed on a rear surface of the base substrate 100 to be disposed under the 3D nano-shell composite 112. However, embodiments of the present invention are not limited thereto. For example, the heating member 150 may be disposed in the trench.

In an embodiment, the 3D nano-shell composite 112 may have a 3D network structure in which nano-scaled pores may be three-dimensionally connected to each other and may be arranged with a periodicity. That is, the 3D nano-shell composite 112 may have a wholly open structure in which substantially all of the pores may be interconnected therein.

Accordingly, efficient mass transfer may be accomplished in the above structure and the surface area of the structure may be maximized such that the pre-concentrator may have increased concentration performance.

Additionally, the 3D nano-shell composite 112 does not consist of a plurality of particles, but may have the network structure so that the 3D nano-shell composite 112 may have uniform and rapid heat transfer coefficient and may have small weight due to high porosity thereof. Therefore, the pre-concentrator may be heated with low energy and may be uniformly heated within short time thereby discharging a gaseous sample with high density and within short time. As a result, the pre-concentrator may have improved concentration performance.

For example, the periodicity of the pores along a horizontal direction in a cross-sectional view may be about 100 nm to about 1,000 nm. However, embodiments are not limited thereto, and the size and the arrangement of the pores may be variously adjusted.

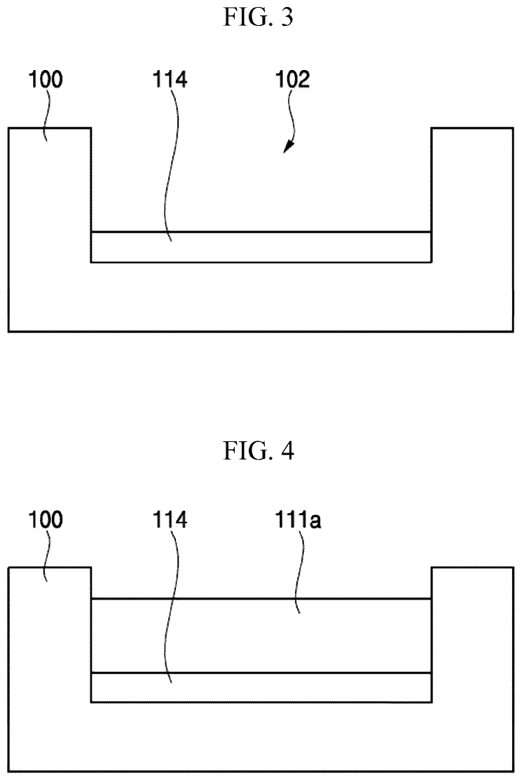

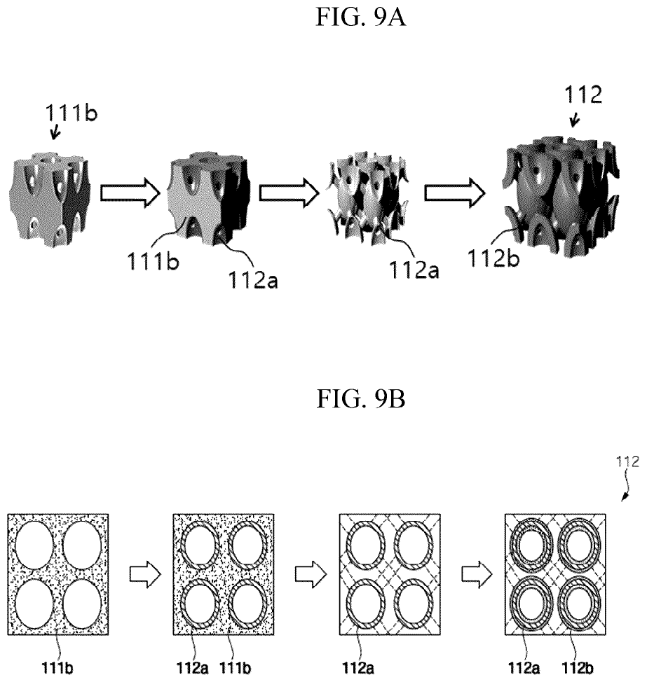

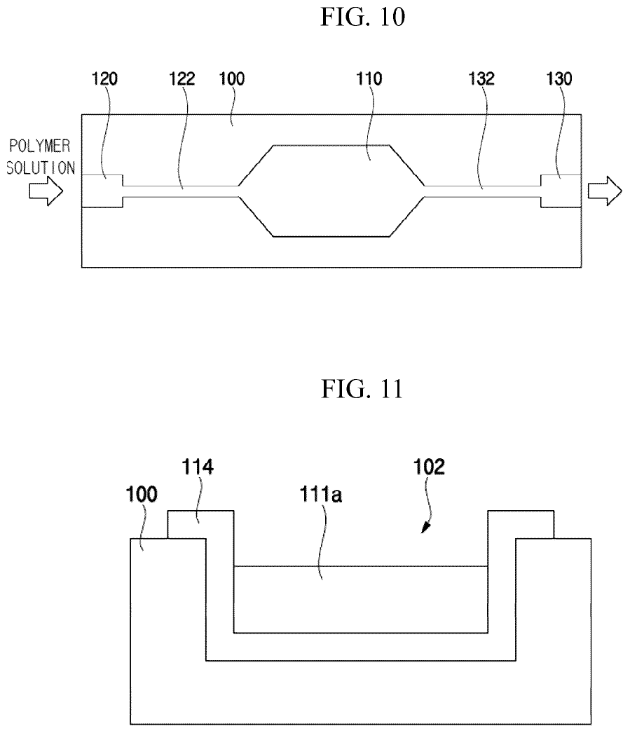

FIGS. 3, 4, 5, 6, 7 and 8 are cross-sectional views illustrating a method of manufacturing a pre-concentrator according to an embodiment of the present invention. FIGS. 3 to 8 are cross-sectional views taken along the line I-I′ of FIG. 1. FIG. 9A is a perspective view schematically illustrating a step of forming a 3D nano-shell composite from a 3D porous template in a method of manufacturing a pre-concentrator according to an embodiment of the present invention. FIG. 9B is a cross-sectional view schematically illustrating a step of forming a 3D nano-shell composite from a 3D porous template in a method of manufacturing a pre-concentrator according to an embodiment of the present invention. FIG. 10 is a plan view illustrating a step of injecting a polymer solution into a trench in a method of manufacturing a pre-concentrator according to an embodiment of the present invention.

Referring to FIGS. 3 and 4, an adhesive film 114 is formed in a trench 102 of a base substrate 100.

The adhesive film 114 includes a polymer. For example, the adhesive film 114 may be formed from a photoresist material. For example, a first photoresist material may be coated in the trench 102 through spin-coating, bar-coating or the like. For example, soft-baking may be performed at about 90° C. to about 100° C. Thereafter, light exposure may be performed by using UV or the like, and hard-baking may be performed on a hot plate at about 100° C. to about 250° C. to form the adhesive film 114.

For example, a thickness of the adhesive film 114 may be about 0.1 μm to about 10 μm, and preferably about 0.5 μm to about 2 μm. When a thickness of the adhesive film 114 is too small, it may be difficult to form a uniform film. When a thickness of the adhesive film 114 is excessively large, a ceramic-polymer composite film may be damaged or deformed after the adhesive film 114 is removed.

Thereafter, a photosensitive film 111a may be formed on the adhesive film 114 in the trench 102.

In an embodiment, a second photoresist material may be coated in the trench 102 through spin-coating, bar-coating or the like, and then soft-baked, for example, at about 60° C. to about 100° C. to form the photosensitive film 111a.

For example, the first photoresist material and the second photoresist material for forming the adhesive film 114 and the photosensitive film 111a may include a same material or different materials. In an embodiment, the first photoresist material and the second photoresist material may include an epoxy based negative-tone photoresist or a DNQ based positive-tone photoresist. In an embodiment, the first photoresist material and the second photoresist material may include an organic or inorganic hybrid material, hydrogel, phenolic resin, or the like.

However, embodiments are not limited thereto, and the adhesive film 114 may be formed from a different material from the photosensitive film 111a. For example, the adhesive film 114 may be formed from a thermosetting resin.

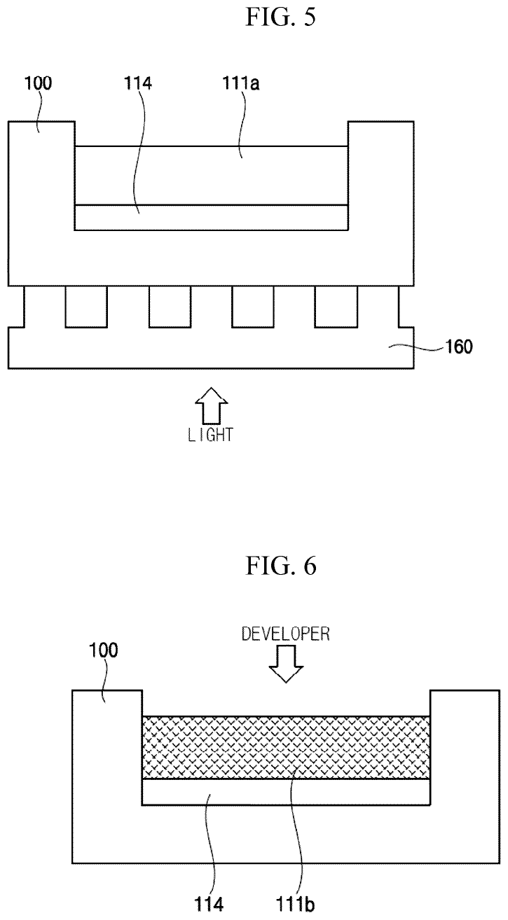

Referring to FIGS. 5 and 6, after a three-dimensionally distributed light is provided to the photosensitive film 111a, the photosensitive film 111a is developed to form a 3D porous template 111b.

In an embodiment, the 3D light-exposure may be performed by proximity-field nano-patterning (PnP) process.

In an embodiment, a phase mask 160 is disposed to contact a rear surface (lower surface) of the base substrate 100, a three-dimensionally distributed light is irradiated onto the photosensitive film 111a through the phase mask 160.

In the PnP process, the photosensitive film 111a may be patterned, for example, by utilizing the periodic 3D distribution of light generated from the interference of light passing through the phase mask 160 including an elastomer material. For example, when the flexible elastomer based phase mask 160 having a concave and convex grid structure on a surface thereof may contact the base substrate 100, the phase mask 160 may be adhered (for example, conformal-contact) to the base substrate 100 by itself because of a Van der Waals force.

When a laser having a wavelength similar to a periodicity of the lattice of the phase mask 60 is irradiated onto the phase mask 160 three-dimensionally distributed light may be formed by Talbot effect. When the photosensitive film 111a is formed from a negative-tone photoresist composition, cross-linking of binders may be selectively caused in a portion where light intensity is relatively high by constructive interference, and may be hardly caused in a remaining portion where light intensity is relatively low. Thus, the remaining portion may be removed in a developing process. Thereafter, a drying process may be performed. As a result, a porous polymeric structure having a 3D network shape with a periodicity of hundreds of nanometers to several micrometers may be formed.

In some example embodiments, pore (channel) size and periodicity in the porous polymeric structure be adjusted by controlling the pattern period of the phase mask 150 and the wavelength of incident light in the PnP process.

The PnP process is disclosed in J. Phys. Chem. B 2007, 111, 12945-12958; Proc. Natl. Acad. Sci. U.S.A. 2004, 101, 12428; AdV. Mater. 2004, 16, 1369 and Korean Patent Publication No. 2006-0109477 (published on Oct. 20, 2006), disclosures of which are incorporated herein by the references.

For example, the phase mask 160 used in the PnP process may include a material such as PDMS (polydimethylsiloxane), PUA (polyurethane acrylate), PFPE (perfluoropolyether), etc.

For example, when the photosensitive film 111a includes the negative-tone photoresist, an unexposed portion of the photosensitive film 111a may remain while an exposed portion thereof may be removed. Thus, the 3D porous template 111b including 3D nano pores may be formed. Examples of a developing solution may include, for example, PGMEA (propylene glycol monomethyl ether acetate).

The 3D porous template 111b may have a 3D network structure, in which nano-scaled pores in a range of about 1 nm to about 2,000 nm are three-dimensionally connected to each other entirely or partially, with a periodicity.

In an embodiment, the 3D porous template 111b may be formed by the above-explained PnP method. However, embodiments of the present invention are not limited thereto. For example, the 3D porous template 111b may be formed by optical patterning methods such as Interference Lithography, Direct Laser Writing, 2-photon Lithography or the like, or physical deposition methods using Colloidal Self-assembly, block-polymer or the like.

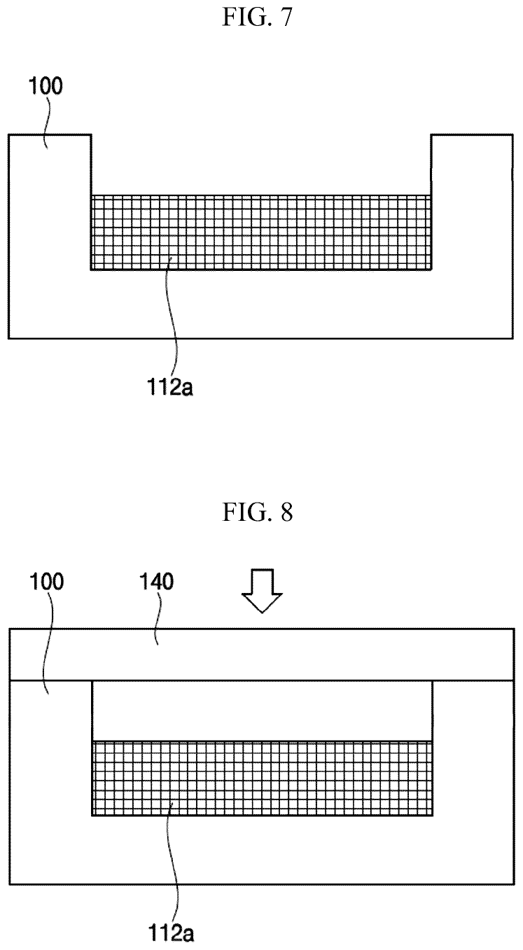

Referring to FIG. 7, a 3D nano-shell composite 112 is formed using the 3D porous template 111b.

In an embodiment, a 3D nano-shell support 112a is formed in pores of the 3D porous template 111b. For example, a thin film is formed on an inner surface of the 3D porous template 111b through a method such as atomic layer deposition, chemical vapor deposition, electroplating, electroless plating or the like. The 3D porous template 111b includes pores connected to each other, and has a small shell thickness. Thus, the thin film extends along the inner wall in the 3D porous template 111b, thereby forming a nano-shell structure in which shell units are three-dimensionally repeated, as illustrated in FIGS. 9A and 9B.

Thereafter, the 3D porous template 111b is removed. Removing the 3D porous template 111b may be performed using a method such as plasma treatment, heat treatment, wet etching or the like.

The thin film is formed with a certain thickness on the inner wall of the pore structure of the 3D porous template 111b to have a shape surrounding the pores in a cross-sectional view. In addition, after the 3D porous template 111b is removed, pores (empty space) are further formed between adjacent shell units. Accordingly, the nano-shell structure may have a high porosity and a large surface area.

Since the adhesive film 114 includes a polymer, it may be removed together when the 3D porous template 111b is removed. After the adhesive film 114 is removed, the 3D nano-shell support 112a may move to a space where the adhesive film 114 is removed, for example, by gravity. However, embodiments of the present invention are not limited thereto. For example, after the adhesive film 114 is removed, an empty space may be maintained between a bottom surface of the trench and the 3D nano-shell support 112a.

Referring to FIG. 8, after the 3D nano-shell support 112a is formed, a cover substrate 140 is coupled to an upper surface of the base substrate 100. Accordingly, the cover substrate 140 may cover the trench.

Thereafter, referring to FIG. 10, by injecting a polymer solution into the trench, the 3D nano-shell support 112a is impregnated with the polymer solution.

The polymer solution may include the previously-described porous polymer and a solvent. In an embodiment, the polymer solution may include Tenax® TA (molecular weight 244.29 g/mol). For example, the solvent may include chloroform, dichloromethane, dichloroethane, toluene, heptane, hexane or the like. In an embodiment, the solvent may include chloroform.

For example, the content of the porous polymer in the polymer solution may be 0.1 wt % to 10 wt %. Preferably, the content of the porous polymer may be 0.5 wt % to 2 wt %. When the content of the porous polymer is too small, it may be difficult to entirely and uniformly coat the 3D nano-shell support 112a, and when the content of the porous polymer is excessive, porosity and uniformity of the 3D nano-shell composite may be reduced.

For example, the polymer solution may be injected through one of the injection part 120 and the discharge part 130, and the polymer solution may be discharged through the other one.

Thereafter, the base substrate 100 is heated to evaporate the solvent of the polymer solution. Accordingly, as shown in FIGS. 9A and 9B, a polymeric layer 112b including a porous polymer is formed on a surface of the 3D nano-shell support 112a. The polymeric layer 112b may surround the 3D nano-shell support 112a. For example, a nano-shell support may be sandwiched between polymeric layers in a cross section of a unit shell.

In an embodiment, a 3D nano-shell composite is used as a stationary phase for gas concentration. Since the 3D nano-shell composite has a network structure, it has a large surface area, and may transfer heat uniformly and fast, and has low weight due to high porosity. Therefore, it is possible to increase adsorption amount of gas. In addition, since the 3D nano-shell composite may be uniformly heated in a short time, it is possible to release gas at a high density in a short time. Thus, it is possible to increase the concentration performance of the micro pre-concentrator.

The micro pre-concentrator may be used for concentration of various gases, and may be effective for concentration of volatile organic compounds, for example.

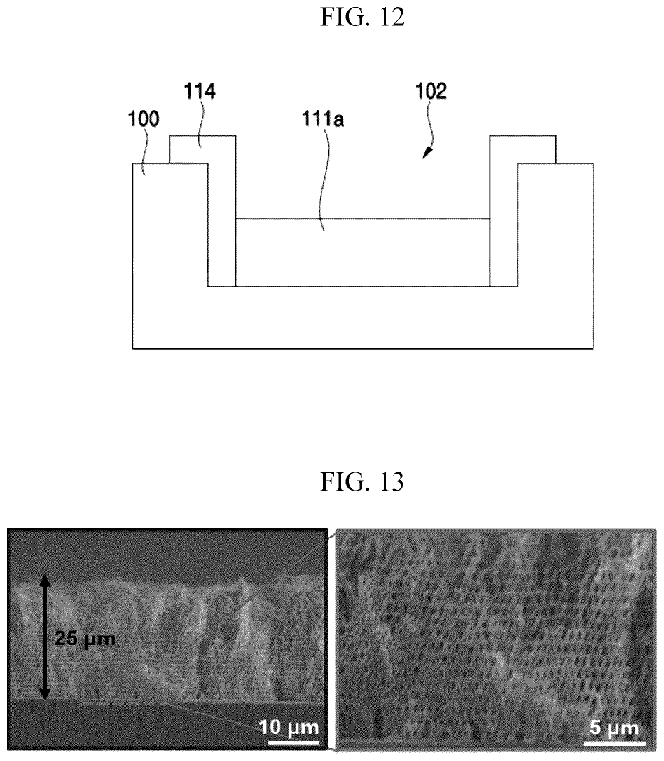

FIGS. 11 and 12 are cross-sectional views illustrating a trench in which an adhesive film and a photosensitive film are formed in a method of manufacturing a micro pre-concentrator according to embodiments of the present invention.

Referring to FIG. 11, an adhesive film 114 may be formed to cover a bottom surface and a side surface of the trench 102. Accordingly, the adhesive film 114 may contact a lower surface and a side surface of a photosensitive film 111a.

Such configuration may increase contact area between the adhesive film 114 and the photosensitive film 111a, thereby increasing adhesion therebetween.

Referring to FIG. 12, an adhesive film 114 may be formed to cover a side surface of the trench 102. That is, the adhesive film 114 may have an opening region overlapping the trench 102. Accordingly, at least a portion of a bottom surface of the trench 102 may not be covered by the adhesive film 114. A lower surface of a photosensitive film 111a may contact the bottom surface of the trench 102, and a side surface of the photosensitive film 111a may contact the adhesive film 114. The adhesive film 114 may extend over the upper surface of the base substrate 100.

In such configuration, the photosensitive film 111a may be directly formed on the bottom surface of the trench 102 while maintaining adhesion with the adhesive film 114. Thus, after the adhesive film 114 is removed, it is possible to prevent the support from being deformed or damaged in the process of descending.

In the embodiment, a 3D nano-shell support may be formed through deposition using a 3D polymeric template. However, embodiments of the present invention are not limited thereto. For example, after filling the 3D polymeric template with metal using plating or the like to form a reversed secondary template, a 3D nano-shell supported is deposited in the secondary template. Such method may allow using a high temperature deposition process.

Hereinafter, the performance of the micro pre-concentrator and its manufacturing method will be described in detail in exemplary embodiments with reference to specific experimental examples. The following experimental examples are merely illustrative, and the embodiments of the present invention are not limited thereto.

### EXAMPLE 1: FABRICATION OF A MICRO PRE-CONCENTRATOR INCLUDING A 3D NANO-SHELL COMPOSITE

1) Fabricating Ordered 3D Porous Template Using PnP Technology

A trench consisting of a concentration part, an injection part, and a discharge part was formed in a glass substrate using a reactive ion etching technique. A photoresist (SU-8 2, Microchem) was coated on the glass substrate through spin coating at 3,500 rpm, followed by soft-baking at 65° C. and 95° C. Thereafter, after exposing the photosensitive film, post-baking was performed to form an adhesive film in the trench.

Thereafter, photoresist (SU-8 50, Microchem) was coated on the glass substrate by spin coating (from 1,000 rpm to 2,500 rpm), and heated at 65° C. for 1 hour and at 95° C. for 24 hours.

Thereafter, a phase mask based on a flexible elastic material with a concavo-convex lattice structure (period: 600 nm, concavo-convex height: 420 nm) on its surface was brought into contact with the lower surface of the glass substrate, and Nd:YAG laser having 355 nm wavelength was irradiated. Thereafter, after performing post-baking, a developing solution (SU-8 Developer, Microchem) was provided to the light-exposed photosensitive film, and dying process was performed to form a 3D porous template.

2) Forming 3D Nano-Shell Support

An alumina (Al₂O₃) thin film was formed in the 3D porous template through atomic layer deposition using trimethylaluminum (precursor) and H₂O (reactant). 1 cycle in the ALD process consisted of 0.5 second for the precursor, 30 seconds for Ar, 1 second for the reactant and 30 seconds for Ar, and 370 cycles were repeated to form an alumina shell having a thickness of 40 nm (deposition speed: 0.11 nm/cycle).

Thereafter, the porous template with alumina deposited thereon was heat-treated in a tube furnace to remove the porous template. The temperature was elevated by 2° C./min, and then kept at 350° C. for 5 hours. Thereafter, the temperature was elevated by 1° C./min, and then kept at 500° C. for 2 hours so that the polymeric template and the adhesive film were removed.

FIG. 13 is a scanning electron microscope (SEM) picture of 3D nano-shell support obtained after removing the polymeric template and the adhesive film in Example 1. Referring to FIG. 13, it can be confirmed that a uniformly ordered nano-shell structure was formed without deformation of an internal structure thereof.

3) Bonding Cover Substrate and Coating Polymer

The glass substrate with the 3D nano-shell support was covered with a silicon cover substrate, and the substrates was bonded through an anodic bonding process.

Thereafter, a chloroform solution containing Tenax TA was injected into the trench with a syringe. Thereafter, the substrate was heated on a hot plate to remove chloroform.

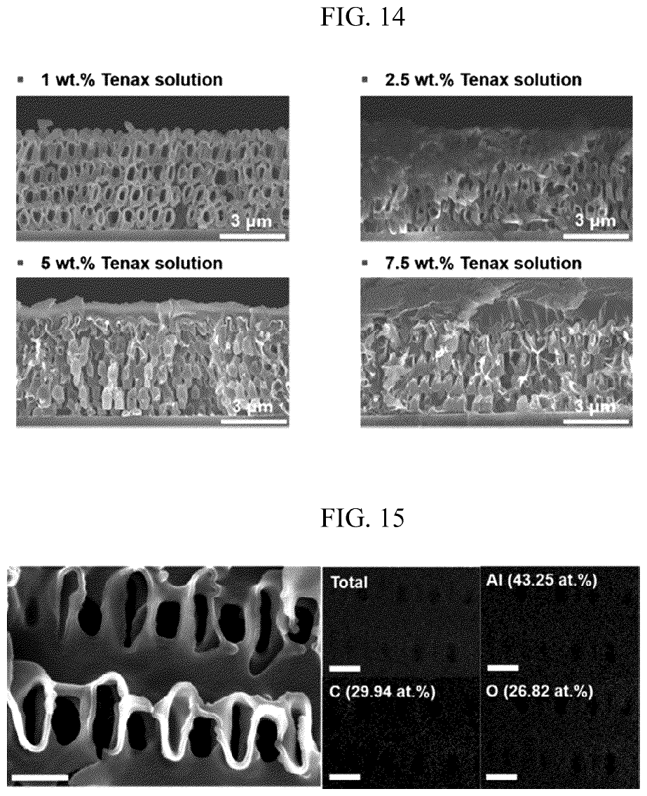

FIG. 14 shows SEM pictures of 3D nano-shell composites obtained according to concentrations of polymer Tenax. Referring to FIG. 14, when the polymer concentration of the polymer solution was excessive, the pores of the 3D nano-shell composite were reduced, or the pore uniformity was reduced.

FIG. 15 is an energy dispersive X-ray spectroscopy (EDS) image of the 3D nano-shell composite (Tenax concentration: 1 wt %) of Example 1. Referring to FIG. 15, it can be confirmed through the carbon (C) content that Tenax was uniformly coated.

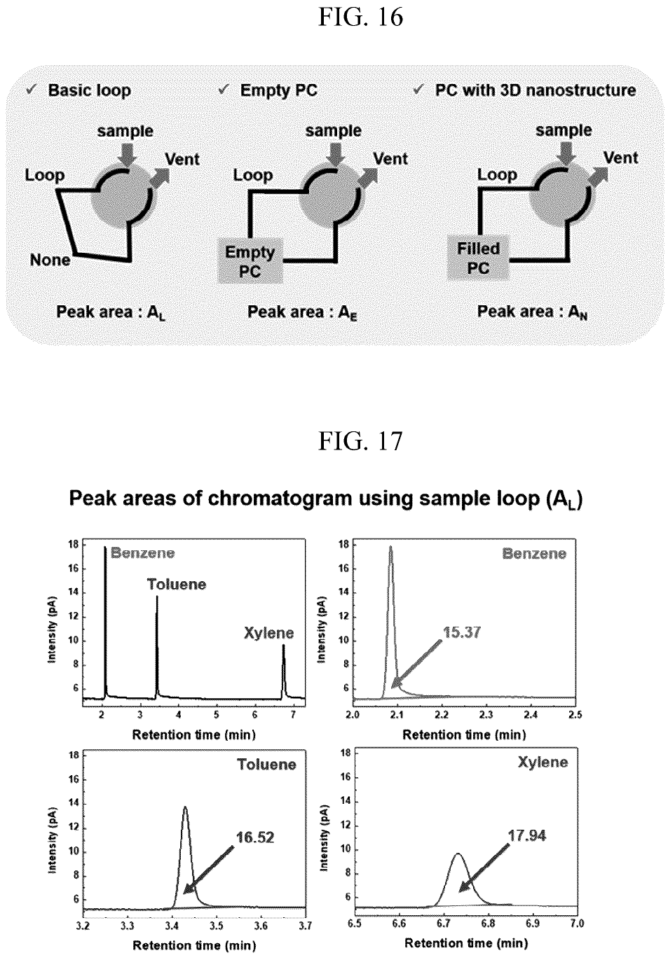

FIG. 16 shows schematic diagrams of an experimental model for evaluating the concentration performance of a micro pre-concentrator according to an embodiment of the present invention.

Additional experiments were conducted to confirm the concentration performance of the micro pre-concentrator according to embodiments of the present invention. In order to exclude influence of a loop in chromatography when measuring concentration ratios, as shown in FIG. 16, a basic loop (Basic loop), an empty trench combined with a loop (Empty PC), and a 3D nanostructure (PC with 3D nanostructure) disposed in a trench combined with the loop were prepared, and a mixed sample of benzene/toluene/xylene was injected for each, and then intensity of chromatography according to the retention time was measured.

The corrected concentration ratio (PF) to exclude influence of the loop is as follows.

$$P F = \frac{{A}_{N} - {A}_{L}}{{A}_{E} - {A}_{L}}$$

(AL: peak area measured through the basic loop, AE: peak area measured through an empty trench , AN: peak area measured through the 3D nanostructure)

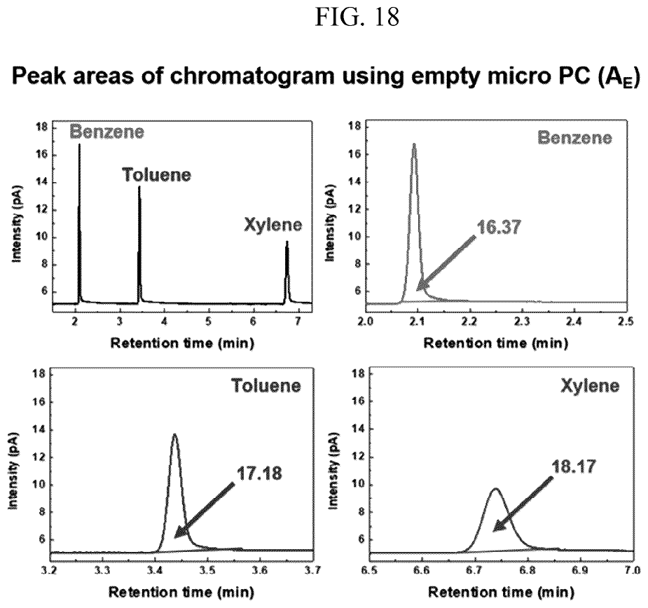

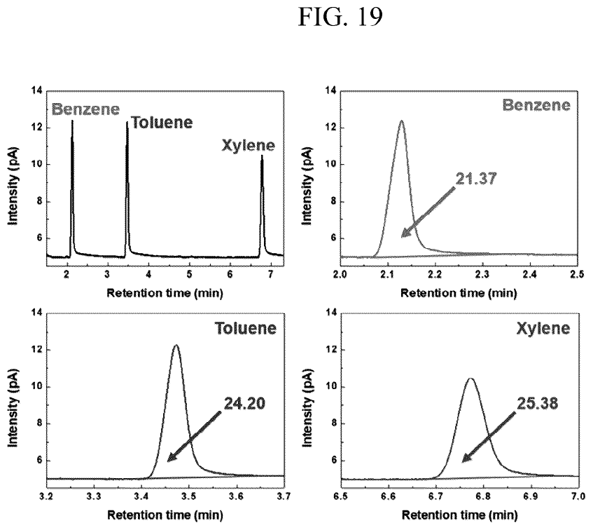

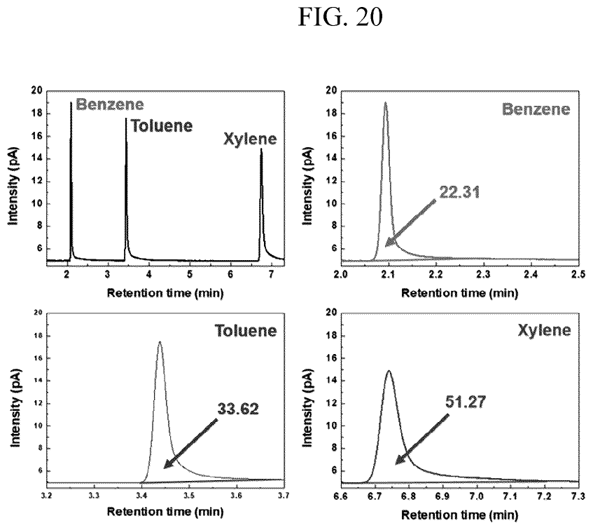

FIG. 17 shows graphs showing chromatographic results and peak areas for mixed samples measured through the basic loop. FIG. 18 shows graphs showing chromatographic results and peak areas for mixed samples measured through the empty trench. FIG. 19 shows graphs showing chromatographic results and peak areas for mixed samples measured through the 3D nano-shell support (thickness: 25 μm) not coated with the polymer in Example 1. FIG. 20 shows graphs showing chromatographic results and peak areas for mixed samples measured through the 3D nano-shell composite (thickness: 25 μm, polymer concentration: 1 wt %) coated with the polymer in Example 1.

FIGS. 17 to 20, the concentration ratios of the 3D nano-shell support without the polymer coating for benzene, toluene, and xylene were 6.00, 11.64, and 32.35, respectively. The concentration ratios of the 3D nano-shell composite having the polymer coating for benzene, toluene, and xylene were 6.94, 25.91, and 144.9, respectively. Thus, it can be confirmed that the concentration performance of the polymer-coated composite structure is excellent.

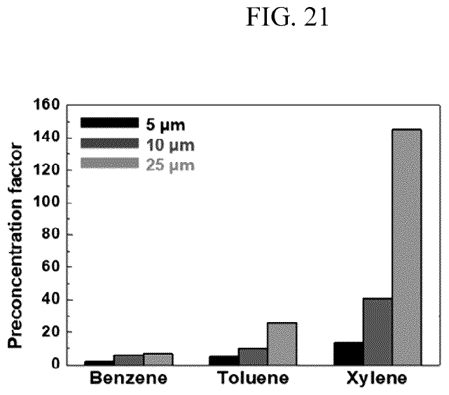

FIG. 21 is a graph showing the concentration ratios of the 3D nano-shell composite coated with the polymer in Example 1 according to the thickness of the 3D nano-shell composite.

Referring to FIG. 21, when the thickness of the 3D nano-shell composite was 20 μm or more, the concentration performance was greatly increased.

The pre-concentrator according to embodiments of the present invention may be used for concentrating or detecting various gaseous components such as volatile organic compounds, explosive compounds or the like.

The foregoing is illustrative of embodiments and is not to be construed as limiting thereof. Although a few embodiments have been described, those skilled in the art will readily appreciate that many modifications are possible in the embodiments without materially departing from the novel teachings and advantages of the present inventive concept. Accordingly, all such modifications are intended to be included within the scope of the present inventive concept as defined in the claims. Therefore, it is to be understood that the foregoing is illustrative of various embodiments and is not to be construed as limited to the specific embodiments disclosed, and that modifications to the disclosed embodiments, as well as other embodiments, are intended to be included within the scope of the appended claims.

What is claimed is:

1. A micro pre-concentrator comprising:
a base substrate having a trench; and
a 3D nano-shell composite disposed in the trench and having pores that are ordered and connected to each other,
wherein the 3D nano-shell composite includes a 3D nano-shell support defined by a thin film extending three-dimensionally and a polymeric layer formed along a surface of the 3D nano-shell support.

2. The micro pre-concentrator of claim 1, wherein a thickness of the 3D nano-shell composite is equal to or more than 20 μm.

3. The micro pre-concentrator of claim 1, wherein a shell thickness of the 3D nano-shell support is 10 nm to 50 nm, and a thickness of the polymeric layer is 50 nm to 100 nm.

4. The micro pre-concentrator of claim 1, wherein the 3D nano-shell support includes a ceramic.

5. The micro pre-concentrator of claim 1, wherein the polymeric layer includes a porous polymer.

6. The micro pre-concentrator of claim 1, further comprising a heating member disposed under the base substrate.

7. A method of manufacturing a micro pre-concentrator, the method comprising:
forming a 3D porous template in a trench of a base substrate;
forming a 3D nano-shell support along a surface of the 3D porous template;
removing the 3D porous template;
coupling a cover substrate to the base substrate such that the cover substrate covers the trench after removing the 3D porous template; and
providing a polymer solution in the trench, after coupling the cover substrate to the base substrate, to form a polymeric layer having a 3D structure along a surface of the 3D nano-shell support.

8. The method of manufacturing a micro pre-concentrator of claim 7, wherein forming of the 3D porous template comprises:
forming an adhesive film, of which at least a portion is disposed in the trench;
forming a photosensitive film in the trench, the photosensitive film contacting the adhesive film;
irradiating a three-dimensionally distributed light onto the photosensitive film through a phase mask; and
developing the photosensitive film.

9. The method of manufacturing a micro pre-concentrator of claim 8, wherein the adhesive film covers an entire portion of a bottom surface of the trench, and the photosensitive film is formed on the adhesive film.

10. The method of manufacturing a micro pre-concentrator of claim 8, wherein the adhesive film covers a side surface of the trench, and a lower surface of the photosensitive film contacts a bottom surface of the trench, and a side surface of the photosensitive film contacts the adhesive film.

11. The method of manufacturing a micro pre-concentrator of claim 7, wherein the 3D nano-shell support is formed through deposition and includes a ceramic.

12. The method of manufacturing a micro pre-concentrator of claim 7, wherein the polymer solution includes 0.5 wt % to 2 wt % of a porous polymer.

13. The method of manufacturing a pre-concentrator of claim 7, wherein a thickness of the adhesive film is 0.5 μm to 2 μm.

14. A micro pre-concentrator comprising:
a base substrate having a trench; and
a 3D nano-shell composite disposed in the trench and having pores that are ordered and connected to each other,
wherein the 3D nano-shell composite includes a 3D nano-shell support, which is defined by a thin film extending three-dimensionally such that shell units are three-dimensionally repeated in the 3D nano-shell support, and a polymeric layer formed along a surface of the 3D nano-shell support to surround the 3D nano-shell support.

15. The micro pre-concentrator of claim 14, wherein a thickness of the 3D nano-shell composite is equal to or more than 20 μm.

16. The micro pre-concentrator of claim 14, wherein a shell thickness of the 3D nano-shell support is 10 nm to 50 nm, and a thickness of the polymeric layer is 50 nm to 100 nm.

17. The micro pre-concentrator of claim 14, wherein the 3D nano-shell support includes a ceramic.

18. The micro pre-concentrator of claim 14, wherein the polymeric layer includes a porous polymer.

19. The micro pre-concentrator of claim 14, further comprising a heating member disposed under the base substrate.

20. The micro pre-concentrator of claim 14, wherein substantially all of the pores are connected to each other such that the 3D nano-shell composite has a wholly open structure.
